(19) United States
(12) Patent Application Publication
(10) Pub. No.: US 2026/0272541 A1
(43) Pub. Date: Sep. 17, 2026

(54) ACTIVE BLOOD PRESSURE FEEDBACK FOR RENAL DENERVATION

(71) Applicant: Boston Scientific Scimed, Inc., Maple Grove, MN (US)

(72) Inventors: Corydon James Carlson, Stillwater, MN (US); Andrew Brian Graveley, Shoreview, MN (US)

(73) Assignee: Boston Scientific Scimed, Inc., Maple Grove, MN (US)

(21) Appl. No.: 19/559,440

(22) Filed: Mar. 6, 2026

Related U.S. Application Data
(60) Provisional application No. 63/770,169, filed on Mar. 11, 2025.

(51) Int. Cl. A61B 18/14; A61B 5/0215; A61B 18/00

(52) U.S. Cl. CPC A61B 18/1492; A61B 5/0215; A61B 2018/0022; A61B 2018/00434; A61B 2018/00511; A61B 2018/00577; A61B 2018/00642; A61B 2018/00994; A61B 2018/1467

(57) ABSTRACT

An example system for renal denervation treatment comprises an ablation catheter including a catheter shaft; an expandable element coupled to the catheter shaft; a plurality of electrode assemblies mounted on the expandable element; an ablation element; and a blood pressure sensor; and a control unit operatively coupled to the ablation catheter, the control unit configured to: measure, via the blood pressure sensor, a baseline blood pressure at or proximate to a target site in a vessel; deliver electrical stimulation through the electrode assemblies at or proximate to the target site; measure, via the blood pressure sensor, a blood pressure response to the delivered electrical stimulation; and selectively deliver ablation energy with the ablation element based on the blood pressure response.

### CROSS-REFERENCE TO RELATED APPLICATION

This application claims the benefit of U.S. Patent Application Serial No. 63/770,169, filed Mar. 11, 2025, entitled " ACTIVE BLOOD PRESSURE FEEDBACK FOR RENAL DENERVATION”, which is incorporated by reference herein in its entirety.

### BACKGROUND

A wide variety of intracorporeal medical devices have been developed for medical use, for example, intravascular use. Some of these devices include guidewires, catheters, and the like. These devices are manufactured by any one of a variety of different manufacturing methods and may be used according to any one of a variety of methods. Of the known medical devices and methods, each has certain advantages and disadvantages. There is an ongoing need to provide alternative medical devices as well as alternative methods for manufacturing and using medical devices. Interventional energy delivery therapies such as renal denervation are typically performed based on the presence of absence of various anatomical landmarks.

### BRIEF SUMMARY

This disclosure provides design, material, manufacturing method, and use alternatives for medical devices. An example method may include a method for treating a patient having high blood pressure. The method may include providing a device. The device may include a catheter extending along a longitudinal axis. A balloon having an unexpanded state and an expanded state may be coupled to an end of the catheter. The balloon may have a plurality of cylindrical treatment zones extending along the longitudinal axis in the expanded state. A plurality of electrode pad assemblies may be mounted to the balloon. Each electrode pad assembly may include a substrate supporting first and second electrode pads with each electrode pad having a pair of elongate bipolar electrodes. The electrode pads of each electrode pad assembly may be longitudinally and circumferentially offset from one another. The method may also include expanding the balloon in the renal artery so as to electrically couple the electrodes with a wall of the renal artery. The method may include driving bipolar energy between the electrodes of each bipolar pair so as to therapeutically alter nerves surrounding the renal artery such that the high blood pressure of the patient is mitigated. Alternatively or additionally, the method may include using the electrically coupled electrodes to detect activity of nerves.

The system and methods herein can be configured for renal denervation treatment and can utilize active (e.g., real-time) in vivo blood pressure feedback mechanisms to improve treatment efficacy and/or safety of the renal denervation treatment. The system comprises a catheter with: i) an expandable balloon containing electrode assemblies, ii) an ultrasound ablation transducer, and iii) a blood pressure sensor (e.g., optically or electrically operated). The system incorporates a central control unit that coordinates imaging, ablation, and monitoring functions to deliver precise therapeutic energy while providing real-time feedback on treatment effectiveness. The treatment process involves measuring baseline blood pressure, stimulating the vessel using electrodes, monitoring blood pressure changes, and delivering controlled ultrasound ablation energy based on the received feedback (e.g., based on changes in blood pressure measured in vivo at or proximate to a target site within the vasculature of a patient). Further, with some embodiments, the treatment may include assessing the vessel condition using an imaging device (e.g., an IVUS imaging device, a optical coherence tomography (OCT) imaging device, or the like). With some embodiments, imaging elements (e.g., an ultrasound transducer or transducers, or the like) configured to capture images are provided along with the ablation elements. In such embodiments, these imaging elements can be utilized to capture images with which to assess the vessel condition.

In an aspect, a system for renal denervation treatment is provided. The system comprising: an ablation catheter including: a catheter shaft; an expandable element coupled to the catheter shaft; a plurality of electrode assemblies mounted on the expandable element; an ablation element; and a blood pressure sensor; and a control unit operatively coupled to the ablation catheter, the control unit configured to: measure, via the blood pressure sensor, a baseline blood pressure at or proximate to a target site in a vessel; deliver electrical stimulation through the electrode assemblies at or proximate to the target site; measure, via the blood pressure sensor, a blood pressure response to the delivered electrical stimulation; and selectively deliver ablation energy with the ablation element based on the blood pressure response.

In some aspects, which may be used in conjunction with or as an alternative to aspects herein, wherein the blood pressure sensor is coupled to an exterior surface of the catheter shaft.

In some aspects, which may be used in conjunction with or as an alternative to aspects herein, wherein the blood pressure sensor is located proximal to the expandable element.

In some aspects, which may be used in conjunction with or as an alternative to aspects herein, wherein the blood pressure sensor is spaced at least three millimeters from a proximal end of the expandable element.

In some aspects, which may be used in conjunction with or as an alternative to aspects herein, wherein the blood pressure sensor is a piezoelectric blood pressure sensor, a capacitive blood pressure sensor, an optical blood pressure sensor, or any combination thereof.

In some aspects, which may be used in conjunction with or as an alternative to aspects herein, wherein the electrical stimulation is configured to induce a transient increase in blood pressure.

In some aspects, which may be used in conjunction with or as an alternative to aspects herein, wherein the electrical stimulation is configured to induce a change in blood pressure.

In some aspects, which may be used in conjunction with or as an alternative to aspects herein, wherein the blood pressure response is determined during a predetermined time period subsequent to delivery of the electrical stimulation, and wherein the blood pressure response is an individual patient specific value that is equal to: an individual blood pressure value measured during the predetermined time period; a highest blood pressure value of a plurality of blood pressure values measured during the predetermined time period; or an average blood pressure value of the plurality of blood pressure values.

In some aspects, which may be used in conjunction with or as an alternative to aspects herein, wherein the control unit is configured to: expand the expandable element at the target site prior to delivery of the electrical stimulation; subsequent to delivery of the electrical stimulation, deflate the expandable element at the target site; and subsequent to deflating the expandable element at the target site, measure the blood pressure response during a predetermined time period.

In some aspects, which may be used in conjunction with or as an alternative to aspects herein, wherein the predetermined time period is about 5 minutes or less.

In some aspects, which may be used in conjunction with or as an alternative to aspects herein, wherein the control unit is configured to: determine a blood pressure difference between the baseline blood pressure and the measured blood pressure response; and compare the blood pressure difference to a blood pressure change threshold.

In some aspects, which may be used in conjunction with or as an alternative to aspects herein, wherein the blood pressure change threshold is equal to at least five millimeters of mercury (mm HG).

In some aspects, which may be used in conjunction with or as an alternative to aspects herein, wherein the control unit is configured to: expand the expandable element at the target site; and initiate delivery of ablation energy through the ablation element responsive to the difference being less than the blood pressure change threshold.

In some aspects, which may be used in conjunction with or as an alternative to aspects herein, wherein the control unit is configured to forego delivery of the ablation energy through the ablation element at the target site responsive to the difference being less than the blood pressure change threshold.

In some aspects, which may be used in conjunction with or as an alternative to aspects herein, wherein the ablation element is an ultrasound transducer.

In another aspect, a system for renal denervation treatment is provided. The system comprising: an ablation catheter including: a catheter shaft; an expandable balloon coupled to the catheter shaft; a plurality of electrode assemblies mounted on the expandable balloon; an ultrasound ablation transducer; a blood pressure sensor; and a control unit operatively coupled to the ablation catheter, the control unit configured to: measure, via the blood pressure sensor, a baseline blood pressure at or proximate to a target site in a vessel; deliver electrical stimulation through the electrode assemblies at or proximate to the target site, wherein the electrical stimulation is configured to induce a change in blood pressure at the target site; measure, via the blood pressure sensor, a blood pressure response subsequent to delivery of the electrical stimulation; determine a blood pressure difference between the baseline blood pressure and the blood pressure response; compare the blood pressure difference to a blood pressure change threshold; and selectively deliver ablation energy, via the ultrasound transducer, based on the comparison of the blood pressure difference to the blood pressure change threshold.

In some aspects, which may be used in conjunction with or as an alternative to aspects herein, wherein the control unit is configured to selectively deliver the ablation energy by: delivering the ablation energy at the target site, responsive to the difference being greater than or equal to the blood pressure change threshold; and forego delivering the ablation energy at the target site, responsive to the blood pressure difference being less than the blood pressure change threshold.

In another aspect, a method for renal denervation treatment is provided. The method comprising: positioning an ablation catheter having an expandable balloon with an ablation element, electrode assemblies, and a blood pressure sensor in a renal artery; measuring a baseline blood pressure of a patient using the blood pressure sensor while the blood pressure sensor is in the renal artery; expanding the balloon to position electrode assemblies against an artery wall of the renal artery; delivering an electrical stimulation with the electrode assemblies to a wall of the renal artery; measuring, via the blood pressure sensor, a blood pressure response of the patient to the electrical stimulation; and selectively delivering ablation energy with the ablation element based on the measured blood pressure response to the electrical stimulation.

In some aspects, which may be used in conjunction with or as an alternative to aspects herein, further comprising: determining a blood pressure difference between the measured blood pressure response of the patient and the baseline blood pressure of the patient; and comparing the blood pressure difference to a blood pressure change threshold, wherein the blood pressure change threshold is equal to at least five millimeters of mercury (mm HG).

In some aspects, which may be used in conjunction with or as an alternative to aspects herein, wherein the ablation energy is selectively delivered by: delivering the ablation energy responsive to the blood pressure difference being greater than or equal to the blood pressure change threshold; and foregoing delivery of the ablation energy responsive to the blood pressure difference being less than the blood pressure change threshold.

The above summary of some embodiments is not intended to describe each disclosed embodiment or every implementation of the present disclosure. The Figures, and Detailed Description, which follow, more particularly exemplify these embodiments.

### BRIEF DESCRIPTION OF THE DRAWINGS

The accompanying drawings, which are incorporated in and constitute a part of this specification, illustrate exemplary embodiments of the present disclosure and together with the description, serve to explain the principles of the disclosure.

FIG. 1A shows a simplified schematic of an example system suitable for providing active blood pressure feedback during renal denervation;

FIG. 1B shows a simplified schematic of a portion of an example ultrasound ablation catheter;

FIG. 2 shows an example flow diagram of active blood pressure feedback for renal nerve denervation; and

FIG. 3 shows an example method for active blood pressure feedback for renal nerve denervation.

### DETAILED DESCRIPTION

For the following defined terms, these definitions shall be applied, unless a different definition is given in the claims or elsewhere in this specification.

All numeric values are herein assumed to be modified by the term “about,” whether or not explicitly indicated. The term “about” generally refers to a range of numbers that one of skill in the art would consider equivalent to the recited value (i.e., having the same function or result). In many instances, the terms “about” may include numbers that are rounded to the nearest significant figure.

The recitation of numerical ranges by endpoints includes all numbers within that range (e.g. 1 to 5 includes 1, 1.5, 2, 2.75, 3, 3.80, 4, and 5).

As used in this specification and the appended claims, the singular forms “a”, “an”, and “the” include plural referents unless the content clearly dictates otherwise. As used in this specification and the appended claims, the term “or” is generally employed in its sense including “and/or” unless the content clearly dictates otherwise.

It is noted that references in the specification to “an embodiment”, “some embodiments”, “other embodiments”, etc., indicate that the embodiment described may include one or more particular features, structures, and/or characteristics. However, such recitations do not necessarily mean that all embodiments include the particular features, structures, and/or characteristics. Additionally, when particular features, structures, and/or characteristics are described in connection with one embodiment, it should be understood that such features, structures, and/or characteristics may also be used in connection with other embodiments whether or not explicitly described unless clearly stated to the contrary.

The following detailed description should be read with reference to the drawings in which similar elements in different drawings are numbered the same. The drawings, which are not necessarily to scale, depict illustrative embodiments and are not intended to limit the scope of the invention.

Physicians use catheters to gain access to and affect therapies by altering interior tissues of the body, particularly within or about the lumens of the body such as blood vessels. For example, balloon angioplasty and other catheters often are used to open arteries that have been narrowed due to atherosclerotic disease.

Catheters can be used to perform renal denervation by ablation treatments (e.g., ultrasound energy treatment) in patients with refractory hypertension. This is a relatively new procedure, which has been found to be clinically effective in treating hypertension. In the procedure, ablation energy such as ultrasound energy is applied to walls of the renal artery to reduce hyper-activation (which is often the cause of chronic hypertension) of the sympathetic nervous system adjacent to the renal artery. This procedure has been found to be successful in some cases, but existing treatments can be both relatively difficult for the physician to accurately perform and quite time-consuming.

Another condition affecting many patients is Congestive Heart Failure (“CHF”). CHF is a condition which occurs when the heart becomes damaged and blood flow is reduced to the organs of the body. If blood flow decreases sufficiently, kidney function becomes altered, which results in fluid retention, abnormal hormone secretions and increased constriction of blood vessels. These results increase the workload of the heart and further decrease the capacity of the heart to pump blood through the kidneys and circulatory system.

It is believed that progressively decreasing perfusion of the kidneys is a principal non-cardiac cause perpetuating the downward spiral of CHF. For example, as the heart struggles to pump blood, the cardiac output is maintained or decreased and the kidneys conserve fluid and electrolytes to maintain the stroke volume of the heart. The resulting increase in pressure further overloads the cardiac muscle such that the cardiac muscle has to work harder to pump against a higher pressure. The already damaged cardiac muscle is then further stressed and damaged by the increased pressure. In addition to exacerbating heart failure, kidney failure can lead to a downward spiral and further worsening kidney function. For example, in the forward flow heart failure described above, (systolic heart failure) the kidney becomes ischemic. In backward heart failure (diastolic heart failure), the kidneys become congested vis-a-vis renal vein hypertension. Therefore, the kidney can contribute to its own worsening failure.

The functions of the kidneys can be summarized under three broad categories: filtering blood and excreting waste products generated by the body's metabolism; regulating salt, water, electrolyte and acid-base balance; and secreting hormones to maintain vital organ blood flow. Without properly functioning kidneys, a patient will suffer water retention, reduced urine flow and an accumulation of waste toxins in the blood and body. These conditions result from reduced renal function or renal failure (kidney failure) and are believed to increase the workload of the heart. In a CHF patient, renal failure will cause the heart to further deteriorate as fluids are retained and blood toxins accumulate due to the poorly functioning kidneys. The resulting hypertension also has a dramatic influence on the progression of cerebrovascular disease and stroke.

The autonomic nervous system is a network of nerves that affect almost every organ and physiologic system to a variable degree. Generally, the system is composed of sympathetic and parasympathetic nerves. For example, the sympathetic nerves to the kidney traverse the sympathetic chain along the spine and synapse within the ganglia of the chain or within the celiac ganglia, then proceeding to innervate the kidney via post-ganglionic fibers inside the “renal nerves”. Within the renal nerves, which travel along the renal hila (artery and to some extent the vein), are the post-ganglionic sympathetic nerves and the afferent nerves from the kidney. The afferent nerves from the kidney travel within the dorsal root (if they are pain fibers) and into the anterior root if they are sensory fibers, then into the spinal cord and ultimately to specialized regions of the brain. The afferent nerves, baroreceptors and chemoreceptors, deliver information from the kidneys back to the sympathetic nervous system via the brain; their ablation or inhibition is at least partially responsible for the improvement seen in blood pressure after renal nerve ablation, or denervation, or partial disruption. It has also been suggested and partially proven experimentally that the baroreceptor response at the level of the carotid sinus is mediated by the renal artery afferent nerves such that loss of the renal artery afferent nerve response blunts the response of the carotid baroreceptors to changes in arterial blood pressure (American J. Physiology and Renal Physiology 279:F491-F501, 2000, the disclosure of which is incorporated herein by reference).

It has been established in animal models that the heart failure condition results in abnormally high sympathetic activation of the kidneys. An increase in renal sympathetic nerve activity leads to decreased removal of water and sodium from the body, as well as increased renin secretion which stimulates aldosterone secretion from the adrenal gland. Increased renin secretion can lead to an increase in angiotensin II levels, which leads to vasoconstriction of blood vessels supplying the kidneys as well as systemic vasoconstriction, all of which lead to a decrease in renal blood flow and hypertension. Reduction in sympathetic renal nerve activity, e.g., via de-innervation, may reverse these processes and in fact has been shown to in the clinic.

As with hypertension, sympathetic nerve overdrive contributes to the development and progression of CHF. Norepinephrine spillover from the kidney and heart to the venous plasma is even higher in CHF patients compared to those with essential hypertension. Chronic sympathetic nerve stimulation overworks the heart, both directly as the heart increases its output and indirectly as a constricted vasculature presents a higher resistance for the heart to pump against. As the heart strains to pump more blood, left ventricular mass increases and cardiac remodeling occurs. Cardiac remodeling results in a heterogeneous sympathetic activation of the heart which further disrupts the synchrony of the heart contraction. Thus, remodeling initially helps increase the pumping of the heart but ultimately diminishes the efficiency of the heart. Decrease in function of the left ventricle further activates the sympathetic nervous system and the renin–angiotensin–aldosterone system, driving the vicious cycle that leads from hypertension to CHF.

Embodiments of the present disclosure relate to a power generating and control apparatus, often for the treatment of targeted tissue in order to achieve a therapeutic effect. In some embodiments, the target tissue is tissue containing or proximate to nerves, including renal arteries and associated renal nerves. In other embodiments the target tissue is luminal tissue, which may further comprise diseased tissue such as that found in arterial disease.

In yet another exemplary embodiment of the present disclosure, the ability to deliver energy in a targeted dosage may be used for nerve tissue in order to achieve beneficial biologic responses. For example, chronic pain, urologic dysfunction, hypertension, and a wide variety of other persistent conditions are known to be affected through the operation of nervous tissue. For example, it is known that chronic hypertension that may not be responsive to medication may be improved or eliminated by disabling excessive nerve activity proximate to the renal arteries. It is also known that nervous tissue does not naturally possess regenerative characteristics. Therefore it may be possible to beneficially affect excessive nerve activity by disrupting the conductive pathway of the nervous tissue. When disrupting nerve conductive pathways, it is particularly advantageous to avoid damage to neighboring nerves or organ tissue. The ability to direct and control energy dosage is well-suited to the treatment of nerve tissue. Whether in a heating or ablating energy dosage, the precise control of energy delivery as described and disclosed herein may be directed to the nerve tissue. Moreover, directed application of energy may suffice to target a nerve without the need to be in exact contact, as would be required when using a typical ablation probe. For example, eccentric heating may be applied at a temperature high enough to denature nerve tissue without causing ablation and without requiring the piercing of luminal tissue. However, it may also be desirable to configure the energy delivery surface of the present disclosure to pierce tissue and deliver ablating energy similar to an ablation probe with the exact energy dosage being controlled by a power control and generation apparatus.

In some embodiments, the efficacy of the denervation treatment can be assessed by measurement before, during, and/or after the treatment to tailor one or more parameters of the treatment to the particular patient or to identify the need for additional treatments.

The system comprises a catheter with an expandable balloon containing electrode assemblies, one or more ultrasound ablation elements (e.g., one or more ultrasound transducers), and a blood pressure sensor. The system incorporates a central control unit that coordinates imaging, ablation, and monitoring functions to deliver precise therapeutic energy while providing real-time feedback on treatment effectiveness. The treatment process involves evaluating vessel conditions using intravascular ultrasound (IVUS), measuring a baseline blood pressure with the blood pressure sensor, stimulating the vessel using the electrode assemblies, measuring (e.g., monitoring) blood pressure changes with the blood pressure sensor, and delivering controlled ultrasound ablation energy based on the received feedback (e.g., based on changes in blood pressure measured in vivo at or proximate to a target site within the vasculature of a patient). In some embodiments, a denervation system may include functionality for assessing whether a treatment has caused or is causing an increase in blood pressure in the vasculature at a target or proximate to a target tissue, which may provide feedback for adjusting parameters of the treatment or indicate the necessity for additional treatments.

While the disclosure focuses on the use of the technology in the vasculature, the technology would also be useful for other luminal tissues. Other anatomical structures in which the present disclosure may be used are the esophagus, the oral cavity, the nasopharyngeal cavity, the auditory tube and tympanic cavity, the sinus of the brain, the arterial system, the venous system, the heart, the larynx, the trachea, the bronchus, the stomach, the duodenum, the ileum, the colon, the rectum, the bladder, the ureter, the ejaculatory duct, the vas deferens, the urethra, the uterine cavity, the vaginal canal, and the cervical canal.

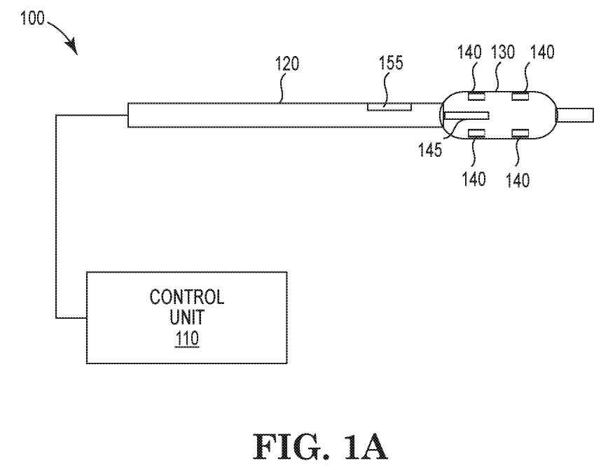

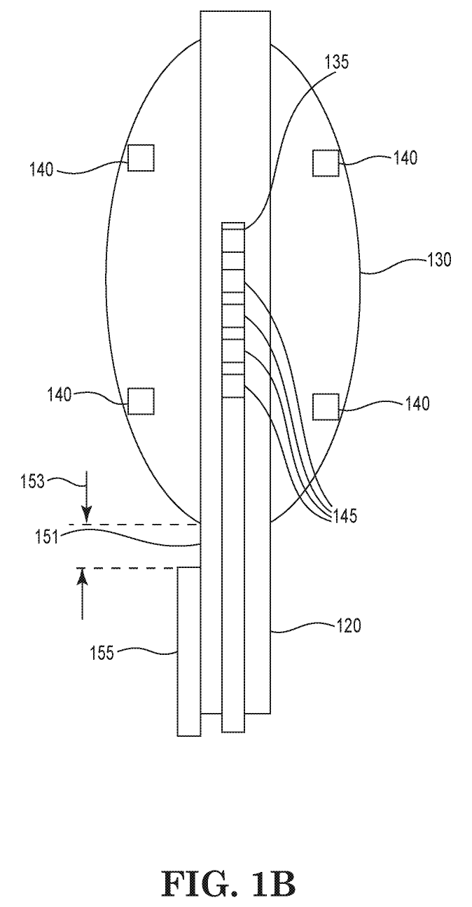

FIG. 1A shows a system 100 for performing a treatment within a body passageway. The system 100 includes a control unit 110, which can include an RF generator for delivering RF electrical energy to an ablation catheter 120. The ablation catheter 120 may be, for example, an ultrasound ablation catheter (e.g., as illustrated in FIG. 1B) that includes an ultrasound transducer configured to convert the RF energy from the RF generator into ultrasound energy for ultrasound ablation. The RF generator and other circuitry of the control unit 110 shown and described for FIG. 1A may be used to generate the nerve stimulation signal and monitor for the response, although, in other embodiments, a separate device may be associated with the system for generating nerve stimulation and/or monitoring response. An exemplary control unit and associated energy delivery methods useable with the embodiments disclosed herein are disclosed in U.S. Patent Number 11,071,583, U.S., Patent Number 9,192,790, U.S. Patent Number 8,473,067, and U.S. Patent Number 10,350,005, the full disclosures of which are incorporated by reference herein. In some embodiments, particularly in some embodiments utilizing monopolar energy delivery, the system may also include a ground / common electrode, which may be associated with the catheter device, a separate pad that is electrically coupled to the control unit 110, or otherwise associated with system 100.

In some embodiments, the control unit 110 may include a processor or otherwise be coupled to a processor to control or record treatment. The processor will typically comprise computer hardware and/or software, often including one or more programmable processor units running machine readable program instructions or code for implementing some, or all, of one or more of the embodiments and methods described herein. The code will often be embodied in a tangible media such as a memory (optionally a read only memory, a random access memory, a non-volatile memory, or the like) and/or a recording media (such as a floppy disk, a hard drive, a CD, a DVD, a non-volatile solid-state memory card, or the like). The code and/or associated data and signals may also be transmitted to or from the processor via a network connection (such as a wireless network, an ethernet, an internet, an intranet, or the like), and some or all of the code may also be transmitted between components of a catheter system and within the processor via one or more buses, and appropriate standard or proprietary communications cards, connectors, cables, and the like will often be included in the processor. The processor may often be configured to perform the calculations and signal transmission steps described herein at least in part by programming the processor with the software code, which may be written as a single program, a series of separate subroutines or related programs, or the like. The processor may comprise standard or proprietary digital and/or analog signal processing hardware, software, and/or firmware, and may desirably have sufficient processing power to perform the calculations described herein during treatment of the patient, the processor optionally comprising a personal computer, a notebook computer, a tablet computer, a proprietary processing unit, or a combination thereof. Standard or proprietary input devices (such as a mouse, keyboard, touchscreen, joystick, etc.) and output devices (such as a printer, speakers, display, etc.) associated with modern computer systems may also be included, and processors having a plurality of processing units (or even separate computers) may be employed in a wide range of centralized or distributed data processing architectures.

In some embodiments, control software for the system 100 may use a client-server schema to further enhance system ease of use, flexibility, and reliability. “Clients” are the system control logic; “servers” are the control hardware. A communications manager delivers changes in system conditions to subscribing clients and servers. Clients “know” what the present system condition is, and what command or decision to perform based on a specific change in condition. Servers perform the system function based on client commands. Because the communications manager is a centralized information manager, new system hardware may not require changes to prior existing client-server relationships; new system hardware and its related control logic may then merely become an additional “subscriber” to information managed through the communications manager. This control schema may provide the benefit of having a robust central operating program with base routines that are fixed; no change to base routines may be necessary in order to operate new circuit components designed to operate with the system.

The ablation catheter 120 can include an expandable element 130, which can be a compliant, non-compliant, or semi-compliant balloon, a plurality of expandable struts, an expandable mesh framework or cage, among other types of expandable elements that are configured to radially expand in vivo. The expandable element 130 includes a plurality of electrode assemblies 140 (e.g., electrodes) electrically coupled to the control unit 110. Such electrode assemblies 140 can be electrically configured to be monopolar or bipolar, as is conventionally known.

One or more electrical conductors of the electrode assemblies 140 may extend at least partially along the catheter shaft and terminate proximate to or within the balloon body. The one or more electrical conductors are configured to conduct electrical energy received from the control unit 110 (e.g., an external energy source in or coupled to the control unit 110) to the one or more fluid conductive regions sufficient to elicit a blood pressure response, as described herein.

In some embodiments, the electrode assemblies 140 may be arranged on the expandable element 130 according to a plurality of cylindrical treatment zones. In other embodiments, the expandable element 130 or other components of the treatment system may include additional electrode assemblies 140 that are not in a treatment zone or are otherwise not used or configured to deliver a treatment or stimulation energy. The expandable element 130 may have a working length of about 10 mm to about 100 mm, or about 18 mm to about 25 mm, which is the approximate longitudinal span of all the treatment zones, among other possible configurations. The electrode assemblies 140 can be attached to a balloon using adhesive, among other possibilities.

In some embodiments, a portion of or the entire ablation catheter 120 can be a single-use device. For instance, the entire ablation catheter 120 can be removably coupled to the control unit 110. The ablation catheter 120 can include one or more ultrasound ablation transducers 145. The ablation catheter 120 can additionally include or one or more imaging transducers 135, as is conventionally known. The imaging transducer 135 can be an intravascular ultrasound (IVUS) transducer, among other types of imaging transducers. As illustrated in FIG. 1B, the imaging transducer can be located distal to each of the ablation transducers on the ablation catheter 120 such being located distal to each of the ultrasound ablation transducers 145. However, other configurations of the imaging transducer(s) and the ablation elements(s) are possible. For instance, while the imaging transducer 135 and the ultrasound ablation transducers 145 are shown as being located on an individual catheter in some embodiments the imaging transducer 135 could be located on an imaging catheter (e.g., an IVUS catheter) and the ultrasound ablation transducers 145 could be located on a separate ultrasound ablation catheter. In such embodiments, the imaging transducer can be disposed at the target site or location prior to, during, and/or subsequent to performing ablation with the ultrasound ablation catheter.

In some embodiments, a cooling arrangement in or coupled to a control unit (e.g., control unit 110, as illustrated in FIG. 1) can be configured for at least one of receiving a thermal transfer fluid from the lumen arrangement and facilitating perfusion of blood passing through the target vessel to cool the expandable element (e.g., a balloon body) during ultrasound ablation of the target tissue (e.g., perivascular tissue).

As mentioned, the ablation catheter 120 can include a blood pressure sensor 155. The blood pressure sensor 155 can be configured to detect a blood pressure in vivo. Suitable blood pressure sensors include various strain gauges or piezoelectric sensors, capacitive sensors, and/or optical sensors. For instance, strain gauges of piezoelectric sensors can be configured to detect mechanical changes in pressure by responding to the deformation caused by blood flow. The force exerted by the blood causes slight bending or stretching of the sensor. This deformation produces a measurable electrical signal proportional to the pressure. Capacitive sensors can be configured to detect blood pressure based on in capacitance. For instance, when the blood pressure varies, it causes a small movement or deformation in the diaphragm (a flexible membrane) of the sensor. The change in the distance between two conductive plates alters the capacitance, which is then converted into a pressure reading. Optical sensors can operate on the principle that changes in blood pressure can alter the way light interacts with or reflects off certain surfaces, which is then converted into an electrical signal which is indicative of a given blood pressure in vivo. An example of a suitable blood pressure sensor is an optical blood pressure sensor such as the optical blood pressure sensor on the Boston Scientific Commet ™ II guidewire.

FIG. 1B shows a simplified schematic of a portion (e.g., distal end portion) of an example of an ultrasound ablation catheter. As illustrated in FIG. 1A and 1B, the blood pressure sensor 155 can be coupled to an exterior surface of the ablation catheter shaft. In some embodiments, the blood pressure sensor 155 can be located at, proximate to, or distal to the expandable element 130. For instance, the blood pressure sensor 155 can be coupled to an exterior surface 151 of the catheter shaft at a location that is proximal to the expandable element 130, as illustrated in FIGS. 1A-1B. Having the blood pressure sensor 155 be located proximal to the expandable element 130 (e.g., rather than on or distal to the expandable element 130) can promote aspects herein. For instance, the blood pressure sensor 155 positioned proximal to the expandable element 130 can permit the blood pressure sensor 155 to readily measure both an initial baseline blood pressure in response to the electrical stimulation and also readily measure a subsequent blood pressure response in response to tissue ablation. For example, the blood pressure sensor 155 located proximal to a location of the expandable element 130 (e.g., is proximal to a proximal end of the expandable element 130) can permit the blood pressure sensor 155 to measure both an initial baseline blood pressure and a subsequent blood pressure response with an absence of lateral movement of the catheter (e.g., with the blood pressure sensor 155 remaining disposed at substantially the same location for measuring both the baseline blood pressure and the blood pressure response). However, in some embodiments the blood pressure sensor 155 can be located distal to the expandable element e.g., depending on a target site in vivo, a type of medical procedure, etc.

In some embodiments, the blood pressure sensor 155 can be spaced a distance 153 from the expandable element 130. For instance, the blood pressure sensor 155 can be spaced at least about one millimeter, at least about two millimeters, at least about three millimeters, at least about 4 millimeters, or at least about 5 millimeters proximal to a proximal end of the expandable element 130. Having the blood pressure sensor 155 be spaced a distance from the proximal end of the expandable element 130 can promote aspects herein, such as ensuring an accurate blood pressure reading due to the blood pressure sensor 155 being upstream (e.g., in terms of blood flow) relative to the position of the expandable element 130. Alternatively, in some embodiments, the blood pressure sensor 155 can be located proximal to and immediately adjacent to (i.e., is not spaced a distance longitudinally from) the expandable element 130.

In some embodiments, the blood pressure sensor 155 can be an individual blood pressure sensor. For instance, the blood pressure sensor 155 can be an individual blood pressure sensor 155 that is located proximal to and spaced a distance from a proximal end of the expandable element 130, as illustrated in FIGS. 1A-1B. However, as mentioned, a location of the blood pressure sensor 155 can be varied. Alternatively or in addition, the blood pressure sensor can be manifested as a plurality of blood pressure sensors. For instance, the blood pressure sensor 155 can be manifested as two or more blood pressure sensors, three or more blood pressure sensors, etc. In some embodiments, the plurality of blood pressure sensors can be manifested as two or more blood pressure sensors that are located proximal to and are spaced differing respective distances from the expandable element 130. Employing a plurality of blood pressure sensors can promote aspects herein such as providing a degree of redundancy (e.g., in the event of a failure or damaged blood pressure sensor), can provide an increased degree of accuracy (e.g., the measured blood pressures herein can be an average of blood pressure measurements from two or more blood pressure sensors, etc.

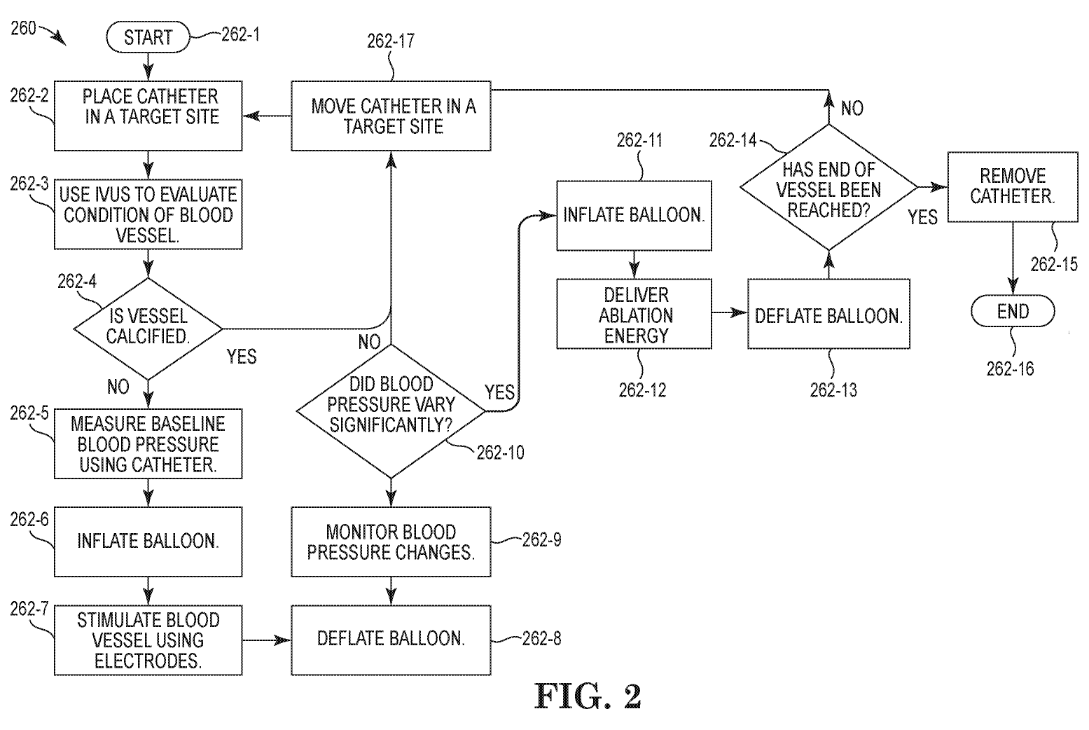

FIG. 2 shows an example flow diagram 260 of active blood pressure feedback for renal nerve denervation. At 262-1, the flow 260 can begin (e.g., responsive to an action by a physician, etc.). At 262-2, the ablation catheter herein can be navigated through a vasculature of a patient to a target site, as is conventionally known. For instance, vascular access can be gained by inserting a catheter through a large vein or artery. A guidewire can be inserted and the ablation catheter can be advanced over the guidewire. In some embodiments, the ablation catheter can include a steering mechanism such as a flexible, steerable tip which can be controlled by the physician through dials or levers on the catheter handle to permit precise directional control. In some embodiments, navigation of the ablation catheter can occur based on various imaging and/or mapping guidance such as fluoroscopy (X-ray), electromagnetic tracking, single, dual, and/or three-dimensional (3D) electroanatomic mapping, intravascular ultrasound, or abdominal ultrasound, etc.

At 262-3, an intravascular ultrasound (IVUS) transducer on the ablation catheter can determine a condition of a target site in a blood vessel. For instance, the condition of a blood vessel wall that is proximate to or adjacent to the expansion element can be determined. In some instances, the IVUS can be an ultrasound probe positioned at a distal end or distal end region of the ablation catheter. The IVUS transducer can emit waves, for instance, into the blood vessel. The sound waves can reflect differently off different types of tissue, for instance, differently off soft plaque versus hard, calcified plaque. The IVUS can thus be configured to determine the thickness and extent of calcification inside the blood vessel (e.g., artery) walls. Alternatively, or in addition, the condition of the blood vessel can be determined based on a degree of physical resistance of longitudinally moving the ablation catheter through a blood vessel, based on optical coherency topographic (OTC), based on Angiography (X-ray with Contrast Dye), and/or can otherwise be determined.

At 262-4, a determination whether a given location inside a blood vessel is calcified is made. For instance, the intravascular ultrasound (IVUS) data indicative of the condition of the blood vessel can be used to determine various aspects pertaining to calcification such as a location and extent (e.g., thickness) of any calcification inside the blood vessels. The determination whether the blood vessel is calcified may be made based on a given thickness, length, and/or surface area associated with any calcification inside of the blood vessel, among other possible factors pertaining to the presence or degree of calcification of the blood vessel. If it is determined that the location inside the blood vessel (e.g., which is proximate or at the same location of the expansion element) is too calcified for effective treatment then the flow can proceed to 262-17. At 262-17, the ablation catheter can be moved distally. The movement of the ablation catheter distally can be responsive to a manual input (e.g., from a physician) or, in some instances, can be automatic. For example, the ablation catheter position can be incremented a predetermined distance (e.g., 1 millimeter, 2 millimeters, 5 millimeters, etc.) automatically by the control unit (e.g., control unit 110 as described with respect to FIG. 1A) responsive to the determination at 262-4 that the location inside the blood vessel is calcified. If it is determined that the location inside the blood vessel is not calcified, then the flow can proceed to 262-5.

At 262-5, the blood pressure sensor can measure a baseline blood pressure in vivo. The blood pressure sensor can be configured to measure a baseline blood pressures sensor that is measured prior to conducting the electrical stimulation described herein (e.g., prior to conducting electrical stimulation to elicit or invoke an increase in blood pressure and prior to conducting tissue ablation). The baseline blood pressure can provide a frame of reference for determining a blood pressure response to electrical stimulation (e.g., sub-perception stimulation and/or stimulation delivered with significantly less energy than required to ablate tissue), as described herein.

At 262-6 the expansion element can be expanded. For instance, the expansion element can be manifested as a balloon, scaffold, or mesh element that is configured to be selectively expanded (e.g., expanded at a target location in vivo subsequent to delivery of the expansion member in an unexpanded condition). For example, the expansion element can be a balloon, as described herein, the balloon is in fluid communication with an inflation lumen of the ablation catheter, as is conventionally known.

At 262-7, an electrical stimulation can be emitted to stimulate a blood vessel. For instance, the electrodes (i.e., electrode assemblies) can be configured to electrically stimulate tissue (e.g., a blood vessel wall) at or near the target site (e.g., a portion of the blood vessel wall). In some embodiments, the electrical stimulation is configured to induce a transient increase in blood pressure. As used herein, a transient increase in blood pressure refers to an increase in blood pressure (relative to the baseline blood pressure) that lasts less than about 1 minute (e.g., before substantially reducing back to the baseline blood pressure). The transient increase in blood pressure can be exhibited substantially immediately (e.g., occurs within 3 minutes or less) following introduction of the electrical stimulation.

The electrical stimulation is provided as a series of rapid AC pulses (milliseconds in length over the period in excess of one minute. The AC frequency may range from 5 to 80 Hz. The electrical stimulation is to induce a change in blood pressure at the target site. For instance, the stimulation energy can be delivered well in excess of the rheobase level to ensure activation of renal nerves.

At 262-8, the expansion element can be deflated or unexpanded. For instance, the expansion element can be manifested as a balloon that is unexpanded at 262-8. The expansion element can be deflated or unexpanded immediately following termination of application of the electrical stimulation or can be deflated or unexpanded a predetermined amount of time subsequent to completion of a last stimulation pulse of the electrical stimulation.

At 262-9, the blood pressure sensor can monitor any changes in blood pressure in vivo. That is, the blood pressure sensor can monitor a blood pressure response to the electrical stimulation at 262-7. For instance, the ablation catheter can perform at least each of the elements described 262-5 (e.g., measure a baseline blood pressure), 262-6 (e.g., inflate a balloon on the ablation catheter), 262-7 (e.g., stimulate the blood vessel with the electrical stimulation using the electrodes on the balloon), 262-8 (e.g., deflate the balloon), and monitor the blood pressure changes at 262-8 while the ablation catheter is at substantially the same location in the blood vessel. Maintaining the ablation catheter at substantially the same location (e.g., the target location) in the blood vessel can permit readily determining any change in blood pressure that is attributable to imparting the electrical stimulation at 262-7. The blood pressure response monitored at 262-9 can be an individual value measured at a given time or can be an aggregate (e.g., average) of a plurality of blood pressure values measured during a time period (e.g., a predetermined time period). In some embodiments, the blood pressure response can be equal to an individual blood pressure value such as an individual blood pressure. In some embodiments, the blood pressure response can be a blood pressure value that is an average of a plurality of blood pressure values that are each measured during a time period (e.g., 1 minute, etc.). However, in some embodiments, the blood pressure response can be equal to a highest blood pressure of the plurality of blood pressure values measure during the time period (e.g., subsequent to emitting the electrical stimulation at 262-7 and subsequent to deflating or expanding the expandable element at 262-8).

At 262-10, a determination of whether the blood pressure response measured at 262-9 deviates significantly from the baseline blood pressure determined at 262-5. For instance, a difference (e.g., an absolute difference) between the baseline blood pressure and the blood pressure response can be determined. This blood pressure difference can be compared to a blood pressure change threshold. The blood pressure change threshold can be equal to a fixed or predetermined value. The blood pressure change threshold can be in a range from about 5 mm HG to about 10 mm HG or about 5 mm HG to about 15 mm HG. The blood pressure change threshold can be equal to at least 3, mm HG, at least mmm HG, at least 5 mm HG, at least 7 mm HG, or at least 10 mm HG. When the blood pressure difference is less than the blood pressure change threshold, it can be determined that the blood pressure difference is not significant. Responsive to determining that the blood pressure difference is not significant (e.g., is insignificant), the flow 260 can proceed to 262-17.

Conversely, when the blood pressure difference is greater than or equal to the blood pressure change threshold, it can be determined that the blood pressure difference is significant and the flow 260 can proceed to 262-1. Notably, a significant blood pressure change can be indicative of the presence of one or more target nerves or tissue which may be amenable to reduction of blood pressure via ablation. Hence, responsive to the determination that the blood pressure difference is significant, the flow 260 can proceed to 262-11. Moreover, it is noted that the determination of the presence of the nerves that are amendable to ablation treatment based on changes in in vivo blood pressure responsive to electrical stimulation herein provides a real-time indication of treatment efficacy. This may prevent the waste of time and energy on treating ineffective areas. This may also ensure that sufficient areas are treated to achieve results. This may also allow the medical staff to evaluate the efficacy of the overall treatment to determine if a patient is likely to have a good outcome or will need additional supporting treatments such as medication.

At 262-11, the expandable element can be expanded. For instance, the expandable element may be a balloon that is expanded (i.e., inflated) to contact the vessel wall at the target location. In some embodiments, the expandable element can be inflated or expanded at substantially the same in vivo location where the blood pressure response was measured at 262-9. Inflating or expanding the expandable element can affix the ablation catheter to the target location within the blood vessel.

At 262-12, the ablation catheter can perform ablation of tissue that is proximate to, or in contact with, the expandable element of the ablation catheter. The ablation can be performed at the target location (e.g., the same location or substantially the same location at which the baseline blood pressure and the blood pressure response were determined). The ablation can be radiofrequency (RF) ablation, ultrasound ablation such as high-intensity focused ultrasound (HIFU), or other possible types of ablation. For instance, an ablation element may be an ultrasound ablation transducer and the ablation may be ultrasound ablation. However, in some embodiments, the ablation element may be a different type, such as an RF electrode, and the ablation treatment may be performed using a different method, such as RF ablation. In some embodiments, when ablation is performed, the expandable element can be positioned substantially at the same in vivo location where the blood pressure response was measured at 262-9. As mentioned, the ablation catheter can include one or more imaging transducers (e.g., the ultrasound imaging transducer 135, as illustrated in FIG. 1B).

At 262-13, the expandable element can be unexpanded (e.g., deflated). For instance, the expandable element may be a balloon which is unexpanded subsequent or responsive to ceasing ablation at 262-11.

At 262-14, a determination can be made whether the end of the blood vessel and/or end of a target site has been reached, as is conventionally known. For instance, the determination can be based on IVUS data indicative of a position of the ablation catheter (e.g., a position of the expandable element) in the blood vessel. Responsive to a determination that the end of the vessel and/or the end of the target site has been reached, the flow 260 can proceed to 262-15 and 262-16 such that the ablation catheter can be removed from the vasculature of the patient and the flow 260 can end. Conversely, responsive to a determination that the end of the vessel and/or the end of the target site has not been reached, the flow 260 can proceed to 262-17. As mentioned, at 262-17 the ablation catheter can be moved distally within the blood vessel either manually or automatically. In any case, subsequent to moving the ablation catheter distally, the flow 260 can return to 262-2 and the continue therefrom.

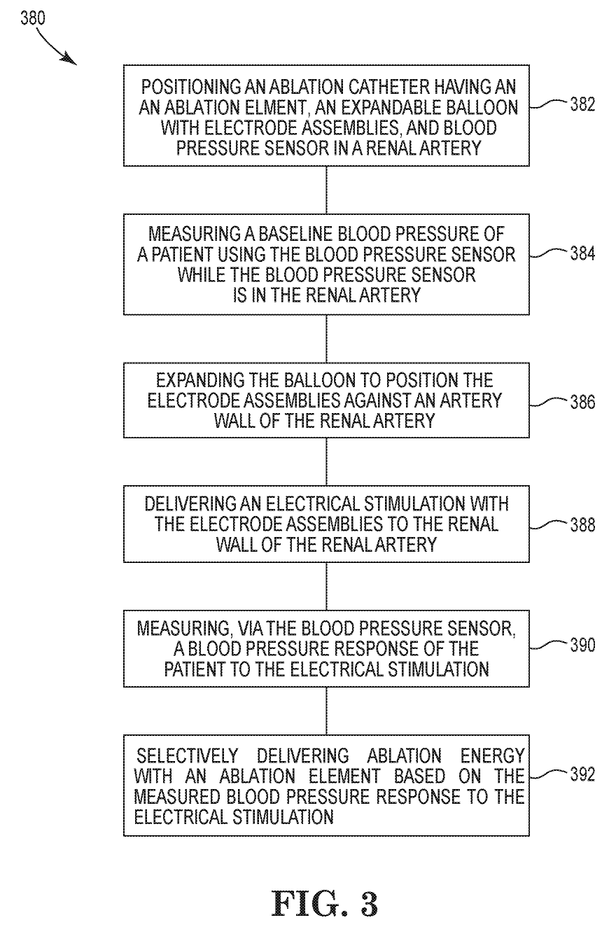

FIG. 3 shows an example method 380 for active blood pressure feedback for renal nerve denervation. At 382, the method 380 includes positioning an ablation catheter having an ablation element (e.g., an ultrasound ablation transducer), an expandable balloon with electrode assemblies and blood pressure sensor in a renal artery, as described herein. For instance, the control unit (e.g., control unit 110) is operationally coupled to the catheter device, which has been placed in a body passageway such that an expandable element (having a plurality of electrode assemblies 140) is placed adjacent to a target site (e.g., section of a body passageway) where therapy is required. Placement of the catheter device at the target site can be performed according to conventional methods, e.g., over a guidewire under fluoroscopic guidance.

In some embodiments, an IVUS imaging transducer in the ablation catheter can emit waves, for instance, into the blood vessel at or near the target site. The sound waves can reflect differently off different types of tissue for instance differently off soft plaque versus hard, calcified plaque, as described herein.

At 384, the method 380 includes measuring a baseline blood pressure of a patient using the blood pressure sensor while the blood pressure sensor is in the renal artery, as described herein. For instance, the blood pressure sensor can measure the baseline blood pressure at the target site subsequent to positioning the ablation catheter at the target site in vivo and subsequent to delivering the electrical stimulation. That is, the blood pressure sensor can measure the baseline blood pressure subsequent to positioning the ablation catheter at the target site in vivo and delivering the electrical stimulation, and prior to delivery of ablation energy. In some embodiments, the blood pressure sensor can measure the baseline blood pressure subsequent to positioning the ablation catheter at the target site in vivo, and prior to expanding the expansion element (e.g., balloon) at the target site.

The baseline blood pressure value refers to an internal blood pressure taken prior to balloon expansion and/or prior to performing ablation with the ablation catheter. For instance, the baseline blood pressure value can be an internal blood pressure measurement by the blood pressure sensor on the ablation catheters herein that is taken subsequent to disposing the balloon at a target site in the section of the body passageway and is taken prior to inflation of the balloon and prior to conducting any ablation with the ablation catheter. Thus, the baseline blood pressure can correspond to a baseline blood pressure at or near the target site for ablation prior to the occurrence of the ablation. Hence, the baseline blood pressure can provide a patient specific reference point for determining any subsequent impact of ablation on blood pressure. Moreover, since the baseline blood pressure is taken in vivo at or proximate to the target site for ablation the value of the baseline blood pressure and any subsequent change (increase therefrom) can be deemed to be at least substantially or entirely attributable to the subsequent ablation of tissue at the target site.

In some embodiments, the baseline blood pressure value is determined over a predetermined time period prior to delivery of electrical stimulation via the ablation catheter. As detailed herein, such electrical stimulation can be configured to induce a change in the blood pressure at or proximate to the target site in a section of a body lumen. For instance, the baseline blood pressure value can be determined within about 30 seconds, within about 1 minute, within about 2 minutes, within about 5 minutes or within about 10 minutes prior to initiating the electrical stimulation (e.g., delivering a first electrical pulse of one or more pulses of the electrical stimulation). In some embodiments, the baseline blood pressure is determined no more than about 10 minutes, no more than about 5 minutes, no more than about 2 minutes, no more than about1 minute, no more than about 30 seconds, no more than about 10 seconds, no more than about 5 seconds, or no more than about 1 second prior to initiating the electrical stimulation.

In some embodiments, the baseline blood pressure is an individual patient specific value (e.g., 85 mm HG) or a range or values (e.g., 72 mm HG to 125 mm HG). For instance, the baseline blood pressure can be a range of blood pressure values extending from the highest and lowest respective blood pressure values monitored over a time period. However, in some embodiments, the baseline blood pressure is an individual blood pressure value such as the highest blood pressure value during the time period or an average of blood pressure values monitored during the time period. For example, the baseline blood pressure can be a highest numerical value out of a plurality of blood pressure values measured at any point during a time period (e.g., within 1 minute) before initiating the electrical stimulation. Similarly, the baseline blood pressure can be equal to an average of some (e.g. excluding one or more statistical outlier) or all blood pressure values monitored during a time period before initiation of the electrical stimulation.

Once placed in at the target site and the baseline blood pressure has been measured, the expandable element 130 can be made to expand, e.g., by pressurizing fluid from 2-10 atm in the case of a balloon. This causes electrode assemblies 140 of the expandable element 130 to come into contact with the body passageway.

At 388, the method 380 includes delivering an electrical stimulation with the electrode assemblies (e.g., on the inflated balloon) to the renal wall of the renal artery. The electrical stimulation can be configured to induce a response for the tissue but not ablate the tissue. For instance, the electrical stimulation can be configured to activate tissue (e.g., via neural stimulation) to induce a corresponding increase in blood pressure without causing damage to the tissue (e.g., without ablating the tissue). In embodiments, a duration of the electrical stimulation can have an intensity, amplitude and/or voltage that is less than the intensity, amplitude and/or voltage associated with tissue ablation, or both. In some embodiments, an intensity of the stimulation energy can be relatively low intensity such as an intensity that is less than about 0.2 watts (W)/centimeter² (cm²), whereas the ablation energy can have an intensity equal to or greater than about 0.5 W/cm².

At 390, the method 380 includes measuring, via the blood pressure sensor, a blood pressure response of the patient to the electrical stimulation, as described herein. For instance, the blood pressure sensor can measure the response of the patient to delivery of the electrical stimulation at a target site immediately following delivery of the electrical stimulation or a predetermined time after initiating or ceasing delivery of the electrical stimulation, among other possibilities. In some embodiments, the blood pressure response is determined during a predetermined time period subsequent to delivery of the electrical stimulation. In some embodiments, the blood pressure response is an individual patient specific value. In some embodiments, the individual patient specific value is equal to: i) an individual blood pressure value measured during the predetermined time period (e.g., at the start, during, or end of the predetermined time period); ii) is a highest blood pressure value of a plurality of blood pressure values monitored during the predetermined time period; or iii) is an average blood pressure value of the plurality of blood pressure values.

At 392, the method 380 includes selectively delivering ablation energy (e.g., ultrasound ablation delivered with an ultrasound transducer) based on the measured blood pressure response to the electrical stimulation, as described herein. For instance, the method 380 can include determining a blood pressure difference between the measured blood pressure response of the patient and the baseline blood pressure of the patient and comparing the blood pressure difference to a blood pressure change threshold (e.g., where the blood pressure change threshold is equal to at least five mm HG, etc.). Hence, the ablation energy can be selectively delivered by either the delivery of the ablation energy at the target site responsive to the difference being greater than or equal to the blood pressure change threshold or forgoing the delivery of the ablation energy at the target site responsive to the difference being less than the blood pressure change threshold. Stated differently, the ablation energy can be selectively delivered at the target site responsive to the difference being greater than or equal to the blood pressure change threshold and foregoing delivery of the ablation energy at the target site responsive to the difference being less than the blood pressure change threshold. Thus, the approaches herein can increase the effectiveness or efficacy of ablation therapy, for instance, as compared to other approaches that do not utilize active (e.g., real-time) in vivo blood pressure feedback prior to conducting ablation treatments (e.g., renal nerve denervation).

Some or all aspects of the flow diagram 260 of FIG. 2 and the method 380 of FIG. 3 can be performed with the system 100 of FIG. 1A. For instance, each aspect of the method of FIG. 3 can be performed with the system 100 of FIG. 1A.

Returning to FIG. 3, after the prescribed therapy at the target site is complete, the expandable element may then be deflated and moved to an untreated section or another target site to repeat the therapy applied in the target site, and any more sections or target sites as needed. The different target sites may be directly adjacent or may be separated by some longitudinal distance.

In some instances, alternative methods other than those illustrated in FIG. 3 will be utilized. For instance, in other embodiments, the treatment will be performed at only a single location in the passageway, and it will not be necessary to move the expandable element to multiple locations in the passageway.

While the exemplary embodiments have been described in some detail, by way of example and for clarity of understanding, those of skill in the art will recognize that a variety of modifications, adaptations, and changes may be employed.

What is claimed is:

1. A system for renal denervation treatment comprising:
an ablation catheter including:
a catheter shaft;
an expandable element coupled to the catheter shaft;
a plurality of electrode assemblies mounted on the expandable element;
an ablation element; and
a blood pressure sensor; and
a control unit operatively coupled to the catheter, the control unit configured to:
measure, via the blood pressure sensor, a baseline blood pressure at or proximate to a target site in a vessel;
deliver electrical stimulation through the electrode assemblies at or proximate to the target site;
measure, via the blood pressure sensor, a blood pressure response to the delivered electrical stimulation; and
selectively deliver ablation energy with the ablation element based on the blood pressure response.

2. The system of claim 1, wherein the blood pressure sensor is coupled to an exterior surface of the catheter shaft.

3. The system of claim 1, wherein the blood pressure sensor is located proximal to the expandable element.

4. The system of claim 1, wherein the blood pressure sensor is spaced at least three millimeters from a proximal end of the expandable element.

5. The system of claim 1, wherein the blood pressure sensor is a piezoelectric blood pressure sensor, a capacitive blood pressure sensor, an optical blood pressure sensor, or any combination thereof.

6. The system of claim 1, wherein the electrical stimulation is configured to induce a transient increase in blood pressure.

7. The system of claim 1, wherein the electrical stimulation is configured to induce a change in blood pressure.

8. The system of claim 1, wherein the blood pressure response is determined during a predetermined time period subsequent to delivery of the electrical stimulation, and wherein the blood pressure response is an individual patient specific value that is equal to:
an individual blood pressure value measured during the predetermined time period;
a highest blood pressure value of a plurality of blood pressure values measured during the predetermined time period; or
an average blood pressure value of the plurality of blood pressure values.

9. The system of claim 1, wherein the control unit is configured to:
expand the expandable element at the target site prior to delivery of the electrical stimulation;
subsequent to delivery of the electrical stimulation, deflate the expandable element at the target site; and
subsequent to deflating the expandable element at the target site, measure the blood pressure response during a predetermined time period.

10. The system of claim 9, wherein the predetermined time period is about 5 minutes or less.

11. The system of claim 9, wherein the control unit is configured to:
determine a blood pressure difference between the baseline blood pressure and the measured blood pressure response; and
compare the blood pressure difference to a blood pressure change threshold.

12. The system of claim 11, wherein the blood pressure change threshold is equal to at least five millimeters of mercury (mm HG).

13. The system of claim 11, wherein the control unit is configured to:
expand the expandable element at the target site; and
initiate delivery of ablation energy through the ablation element responsive to the difference being less than the blood pressure change threshold.

14. The system of claim 11, wherein the control unit is configured to forego delivery of the ablation energy through the ablation element at the target site responsive to the difference being less than the blood pressure change threshold.

15. The system of claim 1, wherein the ablation element is an ultrasound transducer.

16. A system for renal denervation treatment comprising:
an ablation catheter including:
a catheter shaft;
an expandable balloon coupled to the catheter shaft;
a plurality of electrode assemblies mounted on the expandable balloon;
an ultrasound ablation transducer;
a blood pressure sensor; and
a control unit operatively coupled to the ablation catheter, the control unit configured to:
measure, via the blood pressure sensor, a baseline blood pressure at or proximate to a target site in a vessel;
deliver electrical stimulation through the electrode assemblies at or proximate to the target site, wherein the electrical stimulation is configured to induce a change in blood pressure at the target site;
measure, via the blood pressure sensor, a blood pressure response subsequent to delivery of the electrical stimulation;
determine a blood pressure difference between the baseline blood pressure and the blood pressure response;
compare the blood pressure difference to a blood pressure change threshold; and
selectively deliver ablation energy, via the ultrasound ablation transducer, based on the comparison of the blood pressure difference to the blood pressure change threshold.

17. The system of claim 16, wherein the control unit is configured to selectively deliver the ablation energy by:
delivering the ablation energy at the target site, responsive to the difference being greater than or equal to the blood pressure change threshold; and
forego delivering the ablation energy at the target site, responsive to the blood pressure difference being less than the blood pressure change threshold.

18. A method for renal denervation treatment comprising:
positioning an ablation catheter having an expandable balloon with an ablation element, electrode assemblies, and a blood pressure sensor in a renal artery;
measuring a baseline blood pressure of a patient using the blood pressure sensor while the blood pressure sensor is in the renal artery;
expanding the balloon to position the electrode assemblies against an artery wall of the renal artery;
delivering an electrical stimulation with the electrode assemblies to a wall of the renal artery;
measuring, via the blood pressure sensor, a blood pressure response of the patient to the electrical stimulation; and
selectively delivering ablation energy with the ablation element based on the measured blood pressure response to the electrical stimulation.

19. The method of claim 18, further comprising:
determining a blood pressure difference between the measured blood pressure response of the patient and the baseline blood pressure of the patient; and
comparing the blood pressure difference to a blood pressure change threshold, wherein the blood pressure change threshold is equal to at least five millimeters of mercury (mm HG).

20. The method of claim 19, wherein the ablation energy is selectively delivered with the ablation element by:
delivering the ablation energy responsive to the blood pressure difference being greater than or equal to the blood pressure change threshold; and
foregoing delivery of the ablation energy responsive to the blood pressure difference being less than the blood pressure change threshold.
